(19) United States
(12) Patent Application Publication
(10) Pub. No.: US 2025/0375252 A1
(43) Pub. Date: Dec. 11, 2025

(54) INSTRUMENT TRACKER, SYSTEM, AND METHODS OF USE

(71) Applicant: Alphatec Spine, Inc., Carlsbad, CA (US)

(72) Inventors: Lucas Hoffmann, Denver, CO (US); Matthew DiCorleto, Boulder, CO (US)

(73) Assignee: Alphatec Spine, Inc, Carlsbad, CA (US)

(21) Appl. No.: 19/228,657

(22) Filed: Jun. 4, 2025

Related U.S. Application Data
(60) Provisional application No. 63/656,448, filed on Jun. 5, 2024.

(51) Int. Cl. A61B 34/30; A61B 34/20; A61B 90/00

(52) U.S. Cl. CPC A61B 34/30; A61B 90/39; A61B 2034/2055; A61B 2034/2057; A61B 2034/305

(57) ABSTRACT

This disclosure relates to trackers for use in navigated and/or robotic-assisted surgical procedures. A tracker for use in navigated or robotic-assisted surgery comprises a frame connectable to a surgical instrument, and an arm extending from the frame from a first end to a second end. The arm may have a first tracking marker at the second end, where the first tracking marker is for determining a position of the surgical instrument. The tracker may include a second marker disposed on the frame, the second marker for determining a type of the surgical instrument. The tracker may include multiple arms.

### RELATED APPLICATION

The present application claims priority from U.S. Provisional Application No. 63/656,448, filed Jun. 5, 2024, the entirety of which is incorporated herein by reference.

### TECHNICAL FIELD

This disclosure relates generally to trackers for use in navigated and/or robotic-assisted surgical procedures.

### SUMMARY

Disclosed are systems, devices, and/or methods of use thereof regarding trackers for use in navigated and/or robotic-assisted surgical procedures. In various aspects, a tracker for use in navigated or robotic-assisted surgery includes a frame connectable to a surgical instrument, and at least one arm extending from the frame from a first end to a second end. The at least one arm may have a surgical instrument positioning tracking marker at the second end, where the surgical instrument positioning tracking marker is for determining a position of the surgical instrument. The tracker may also include an instrument-type tracking marker disposed on the frame, where the instrument-type tracking marker is for determining a type of the surgical instrument.

In various aspects, a system for use in a navigated or robotic-assisted surgical procedure includes a tracker connectable to a surgical instrument for use in the surgical procedure. The tracker may be for identifying a type of the surgical instrument and for tracking a position of the surgical instrument during the surgical procedure. The tracker may include a frame connectable to the surgical instrument and at least one arm extending from the frame from a first end to a second end. The at least one arm may have a first tracking marker at the second end, where the first tracking marker is for determining a position of the surgical instrument. The tracker may further include a second tracking marker in connection with the frame, where the second tracking marker is for determining a type of the surgical instrument. The system may also include a processor in communication with a sensor configured to track the tracker and a display in communication with the processor. The processor is for receiving signals from the sensor and for determining the type of the surgical instrument and the position of the surgical instrument during the surgical procedure. The display may be for displaying the type of the surgical instrument and the position of the surgical instrument during the surgical procedure.

In various aspects, a method for identifying a type of surgical instrument in navigated or robotic-assisted surgery includes connecting a tracker to a surgical instrument and positioning an instrument-type tracking marker of the tracker in a first position. The method may also include detecting the instrument-type tracking marker at the first position, with the first position corresponding to a first type of surgical instrument. The method may further include determining the type of the surgical instrument based on the detected first position of the instrument-type tracking marker. For a second type of surgical instrument different than the first type, the method may include the step of positioning an instrument-type tracking marker of the tracker in a second position, with the second position corresponding to a second type of surgical instrument.

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1 illustrates an exploded view of a tracker for identifying a type of surgical instrument in a navigated or robotic-assisted surgical procedure;

FIG. 2 illustrates a front, perspective view of a frame of the tracker of FIG. 1;

FIG. 3 illustrates a back, perspective view of the frame;

FIG. 4 illustrates a front, perspective view of a center of the tracker of FIG. 1;

FIG. 5 illustrates a back, perspective view of the center;

FIG. 6 illustrates an assembled view of the tracker, where the center has been received by the frame;

FIG. 7 illustrates a side, cross-sectional view of the tracker of FIG. 6 taken through the line 7-7 of FIG. 6;

FIG. 8 illustrates a side, perspective, cross-sectional view of the tracker of FIG. 7;

FIG. 9 illustrates a front, perspective, cross-sectional view of the tracker of FIG. 6 taken through the line 9-9 of FIG. 6;

FIG. 10 illustrates an exploded view of one embodiment of a tracking system, including the tracker of FIGS. 1 through 9, and a mechanism for connecting the tracker to a surgical instrument;

FIG. 11 illustrates a side view of the tracking system of FIG. 10;

FIG. 12 schematically illustrates another embodiment of a tracking system;

FIG. 13 is a flowchart of one example method of identifying a type of surgical instrument in navigated or robotic-assisted surgery;

FIGS. 14A and 14B illustrate additional embodiments of a tracker; and

FIGS. 15A and 15B illustrate examples of engagement mechanisms for the tracker of FIGS. 1 through 12 or FIG. 14A.

### DETAILED DESCRIPTION

During surgical procedures, precise navigation of surgical tools is important to minimize risks of errant drilling, cutting, or placement of surgical components within a patient (e.g., screws, pins, etc.). Image-guided or other navigation assistance within a surgical procedure can help minimize these risks. Various instruments that are desired to be tracked may be used during an operative procedure. Image data is generally acquired, either intra-operatively or pre-operatively, and the instrument is generally illustrated, and superimposed on the captured image data to identify the position of the instrument relative to the patient space. Therefore, the instrument may include tracking sensors that may be detected by a suitable tracking system, such as an optical tracking system or other type of tracking system.

During surgical navigation, image data of the patient can be correlated with the architecture of various surgical instruments. Typically, each instrument is registered with the surgical navigation system such that when the surgical navigation system detects the instrument, the registration and the architecture of the instrument are matched. During a procedure, however, the medical professional may need to change the orientation of the instrument, his or her orientation relative to the instrument and/or add or remove components to/from the instrument during the procedure. Typically, each of the above changes to the instrument requires an additional registration so that the new instrument configuration is registered with the surgical navigation system.

For example, certain instruments can receive additional components during a medical procedure. The instrument can have a first configuration that defines a bare instrument, i.e., no additional components attached to the instrument. The first configuration can be stored in the surgical navigation system. A component can be added to the instrument, which requires a re-registration so the architecture of the component in addition to the architecture of the instrument is registered with the surgical navigation system. As each configuration of the instrument changes, the instrument's new configuration must be re-registered.

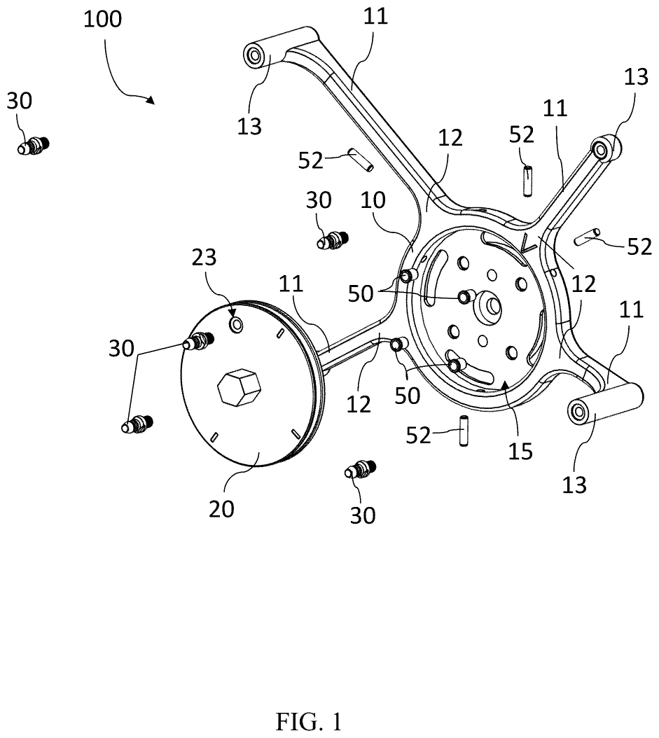

FIG. 1 illustrates an exploded view of a tracker 100 for identifying a type of surgical instrument in a navigated or robotic-assisted surgical procedure. The tracker 100 includes a frame 10 and a tracker center 20 (also referred to herein as “the center 20”) connectable to the frame 10. The frame 10 includes one or more (e.g., a plurality) of arms 11 extending from the frame 10. Each arm 11 extends from a first end 12 to a second end 13. At the second end 13, each arm 11 may receive a tracking marker 30. The center 20 also has a tracking marker 30 in connection therewith, and may define a void 23 for receiving the tracking marker 30. In some embodiments, the tracker may be substantially the form factor illustrated in FIG. 1. In other embodiments the tracking marker 30 shown in FIG. 1 can have a larger tracker attached thereto (i.e., a large sphere for an optical tracker, etc.). The embodiments shown in the figures generally have four arms on the frame 10, but any number of arms desired can be used.

The tracking markers 30 may be optical tracking markers, thermal tracking markers, radiofrequency tracking markers, or any appropriate type of tracking marker desired. The tracking markers 30 at the second end of each arm 11 are for tracking a position of a surgical instrument attached to the tracker 100—these tracking markers 30 may be referred to as surgical instrument positioning tracking markers. The tracking marker 30 in connection with the center 20 is for determining a type of surgical instrument attached to the tracker 100—this tracking marker 30 in connection with the center 20 may be referred to as an instrument-type tracking marker or instrument-type marker. As discussed more below, a position of the instrument-type marker 30 relative to the frame 10 may correspond to a type of surgical instrument attached to the tracker 100. The tracking markers 30 and/or a position thereof may be detected by a detector, which may communicate with a processor to (i) determine a position of the surgical instrument and/or (ii) determine a type of the surgical instrument attached to the tracker 100.

The frame 10 also includes or defines a central recess 15 for receiving the center 20. The central recess 15 also receives a plurality of components for fastening the center 20 and facilitating movement of the center 20 within the central recess 15. For example, the central recess 15 may receive a plurality of spring plungers 50 to facilitate rotation/movement of the center 20 within the central recess 15 when the center 20 is received within the central recess 15. The central recess 15 may also receive a portion of one or more dowels 52 for securing the center 20 within the central recess 15.

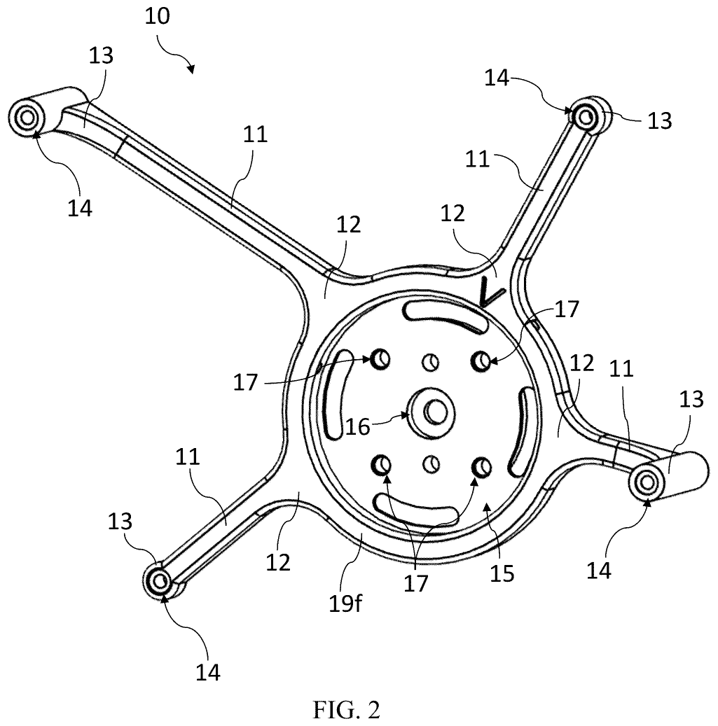

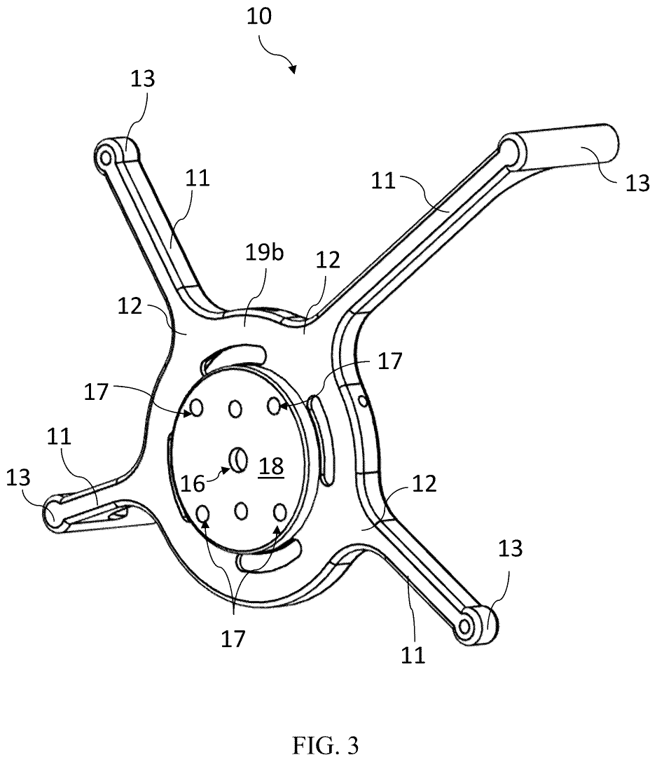

FIGS. 2 and 3 illustrate the frame 10 of the tracker 100 of FIG. 1. As before, the frame 10 includes a plurality of arms 11, each extending from the first end 12 to the second end 13. Defined in the second end 13 is void 14 for receiving a surgical instrument positioning tracking marker 30. The void 14 may be threaded to match and engage with threading of the tracking marker 30. Alternatively, the tracking marker 30 may be press- or friction-fit within the void 14. Additionally, and/or alternatively, the tracking marker 30 may snap or slip into the void 14 or otherwise connect to the second end 13 of the arm 11.

The central recess 15 defines a plurality of apertures 17 for receiving the spring plungers 50. The central recess 15 also defines a hole 16 for receiving a fastener to attach the tracker 100 to a surgical instrument. For example, as seen in FIG. 3, the back 19b of the frame 10 includes a platform 18 for facilitating connection or attachment of the tracker 100 to the surgical instrument. The hole 16 extends from the front 19f of the frame 10 (e.g., the front of the central recess 15) to the back 19b. A fastener will correspondingly extend through the hole 16, from the front 19f to the back 19b, and be accessible through the platform 18. Additional cut-outs, windows, or apertures may be defined in the central recess 15 to remove excess material from the tracker 100 and decrease a weight of the tracker 100.

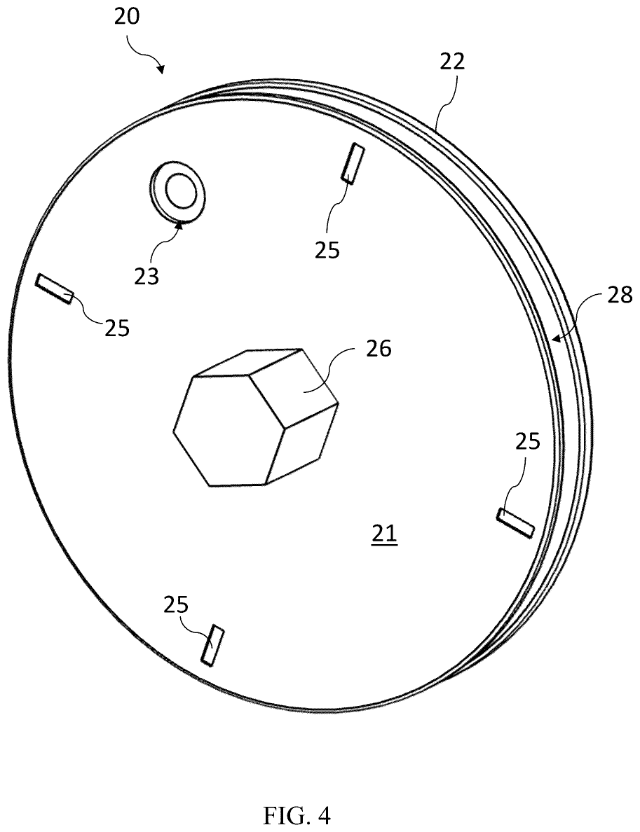

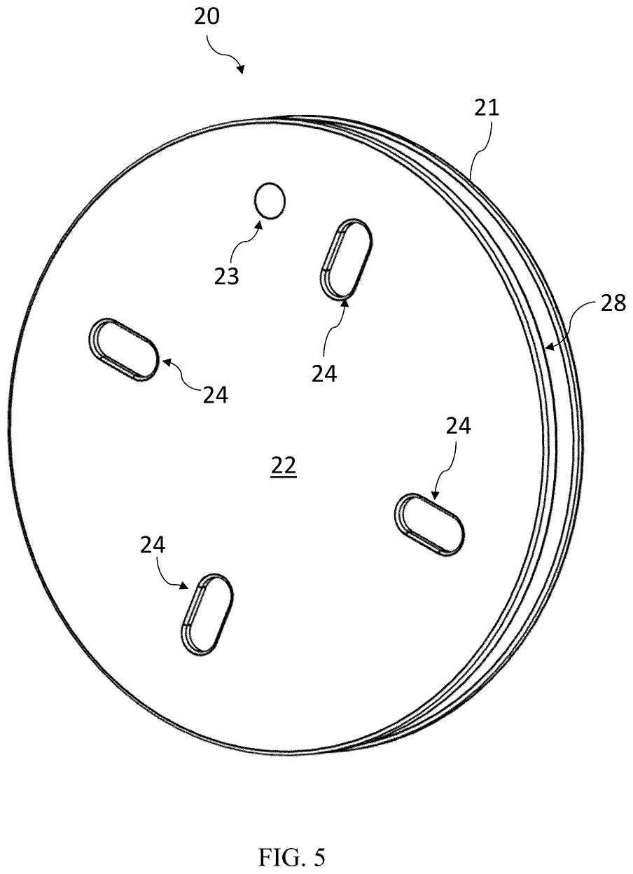

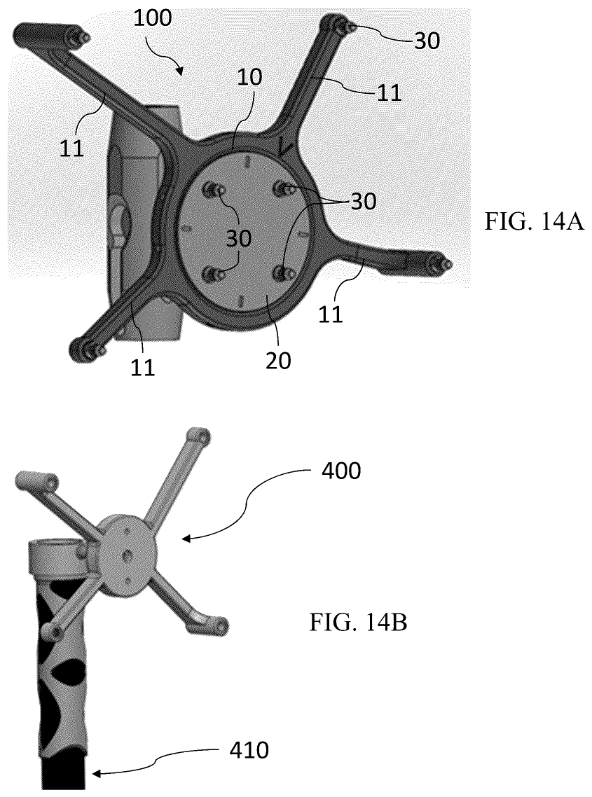

FIGS. 4 and 5 illustrate the center 20 of the tracker 100 of FIG. 1. The center 20 includes a front surface 21 and a back surface 22, with a circumferential groove or channel 28 defined therebetween. Defined in the front surface 21 and extending through to the back surface 22 is the void 23 for receiving the tracking marker 30 (i.e., the instrument-type tracking marker 30). The center 20 and/or the frame 10 may include more than one instrument-type marker 30. For example, referring to FIG. 14A, four (4) instrument-type markers 30 may be in connection with the center 20. Including more than one instrument-type tracking marker 30 may allow multiple surgical instruments to be quickly and readily determined. In other embodiments, these instrument-type markers 30 can allow another larger marker (such as a spherical optical marker) to be connected to the markers 30. Depending on the type of marker 30 used, it may be either integrated with marker 30 and/or attached to marker 30.

Referring briefly to FIG. 14B, the tracker 100 may not include a center 20 and the instrument-type tracking marker 30 may be in association with the frame 10. This illustrated embodiment of a tracker 400 is configured to be permanently secured to an instrument 410, such as a powered drill. In some embodiments, securing the tracker 400 to the instrument 410 may require portions of the instrument 410 to be removed-such as a cap or end piece-to allow the tracker 400 to be slid onto the instrument 410 and possibly welded onto the instrument 410.

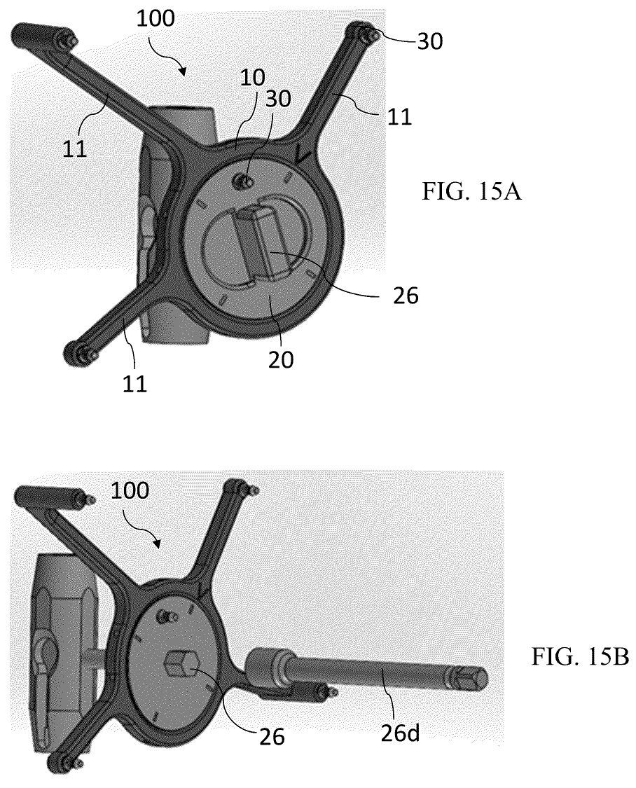

Extending from the front surface 21 is an engagement mechanism 26. The engagement mechanism 26 facilitates positioning of the center 20 within the central recess 15 and the frame 10. The engagement mechanism 26 may be a handle to be physically grasped and manipulated by a user (e.g., a practitioner utilizing the tracker 100 within a surgical procedure, see FIG. 15A). Additionally, and/or alternatively, the engagement mechanism 26 may be a nut to be engaged and manipulated by a driver (see FIG. 15B and driver 26d). In some embodiments, the instrument-type tracking marker 30 may not be detected by a detector until the engagement mechanism has been manipulated and the instrument-type tracking marker 30 has been positioned.

The center 20 may have one or more discrete positions about the frame 10. Concomitantly, the instrument-type tracking marker 30 in connection with the center 20 may have one or more discrete position about the frame 10. Each discrete position may correspond to a different type of surgical instrument attached to the tracker 100. More specifically, a position of the instrument-type marker 30 relative to the frame 10 may correspond to the type of surgical instrument attached to the tracker 100. The center 20 may include indicia or other indicators 25 corresponding to each discrete position. In some embodiments, the center 20 may have two (2), three (3), four (4), or more discrete positions, with each position corresponding to a discrete type of surgical instrument. For example, a first position corresponds to a first type of surgical instrument; a second position corresponds to a second type of surgical instrument; a third position corresponds to a third type of surgical instrument; and so on.

When there are four (4) discrete positions, the discrete positions may be spaced apart from each other by a distance of about 90-degrees. Each discrete position may have a tolerance or threshold value of about 10-degrees. That is, the center 20 and/or the instrument-type tracking marker 30 may be positioned within 10-degrees of a discrete position and the type of surgical instrument can still be determined based on the discrete position. The tolerance or threshold value can be adjusted as desired. For example, in systems where the tracker is in communication with software, the tolerance can be adjusted via software. As discussed with respect to FIGS. 1 and 2, the center 20 is positioned within the central recess 15 and movement of the center 20 is facilitated by one or more spring plungers 50. Referring briefly to FIG. 5, the back surface 22 of the center 20 defines one or more voids or indentations 24 for interfacing and engaging with the one or more spring plungers 50.

Though the center 20 has been discussed in relation to rotational movement, other movements of the center 20 and/or the instrument-type tracking marker 30 relative to the frame 10 are contemplated herein. For example, the instrument-type tracking marker 30 may slide along the center 20. Alternatively, the instrument-type tracking marker 30 may be in connection with the frame 10 rather than the center 20. For example, the instrument-type tracking marker 30 may be in connection with the central recess 15 and may be moveable within the central recess 15.

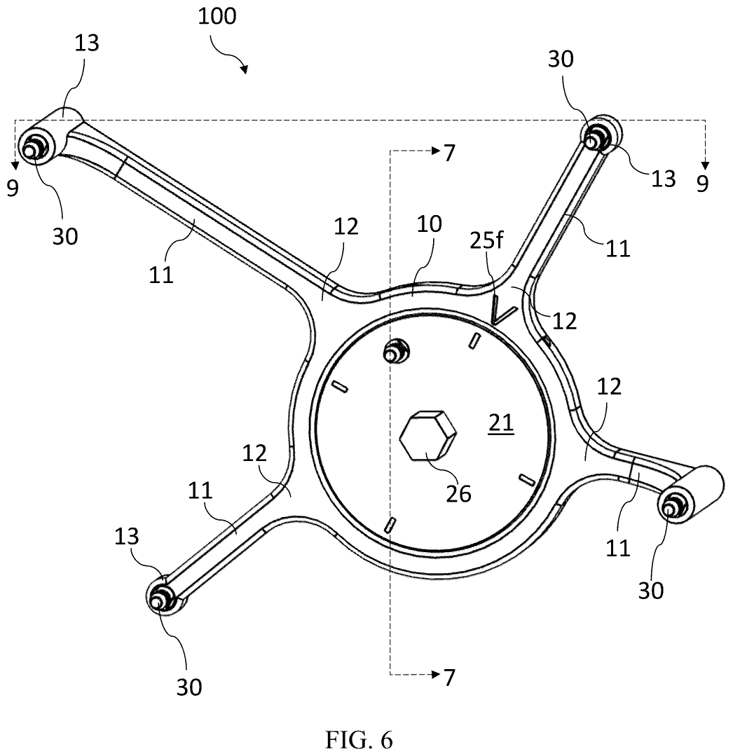

FIG. 6 illustrates an assembled view of the tracker 100, where the center 20 has been received by the frame 10. When the center 20 is received within the central recess 15, the back surface 22 of the center 20 abuts the central recess 15. Additionally, the spring plungers 50 are engaged by the indentations 24. The frame 10 may include indicia 25f for alignment with the indicia 25 of the center 20 during positioning of the instrument-type marker 30 about the frame 10. In some embodiments, the indicia 25f corresponds to a zero-or home-position for the instrument-type tracking marker 30.

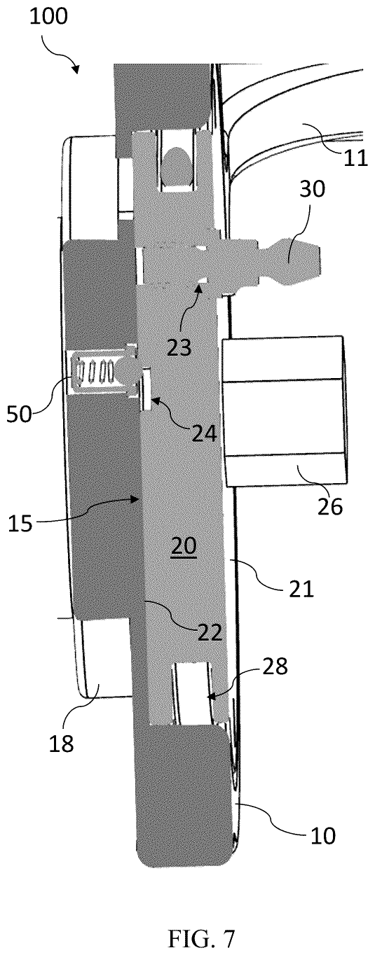

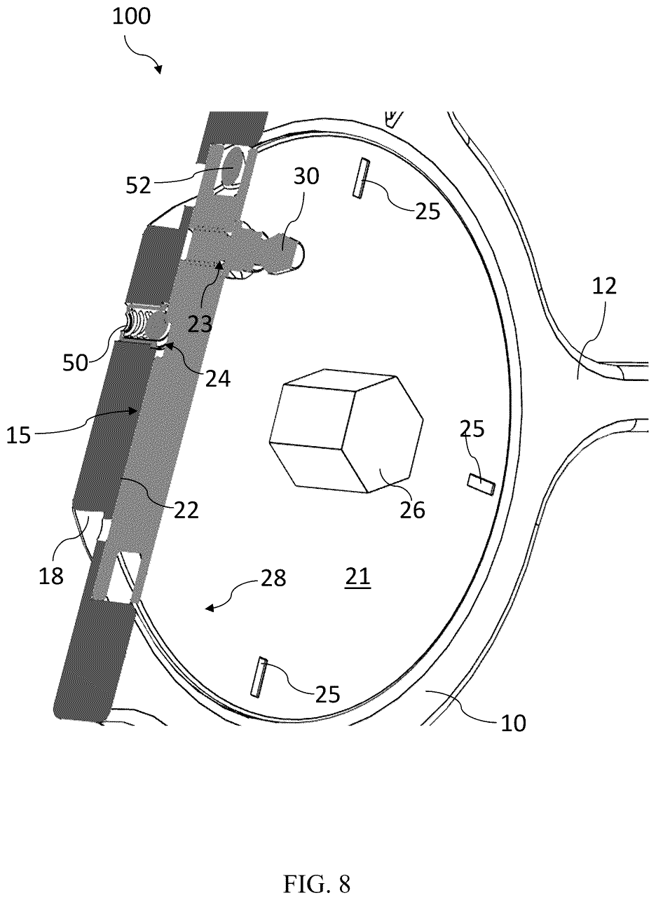

FIGS. 7 through 9 illustrate various cross-sectional views of the tracker 100 of FIG. 6. FIGS. 7 and 8 illustrate cross-sectional views of the tracker 100 taken along the line 7-7. As illustrated, the spring plunger 50 can be seen interfacing and engaging with an indentation 24. The spring plunger 50 may be a ball spring plunger 50, which facilitates rotation or other movement of the center 20 (and, thus, the instrument-type tracking marker 30) about and relative to the frame 10.

The ball of the spring plunger 50 may sit in or engage the indentation 24 when the center 20 is in one or more discrete positions about the frame 10. A spring of the spring plunger 50 may bias the ball towards the back surface 22 and the indentation 24 of the center 20. Rotational or other forces applied to the center 20 cause the ball to disengage from the indentation 24 and retract into the plunger 50, compressing the spring. Upon placement of the center 20 in a discrete position, the compressed spring will expand, causing the ball to re-engage an indentation 24 of the center 20.

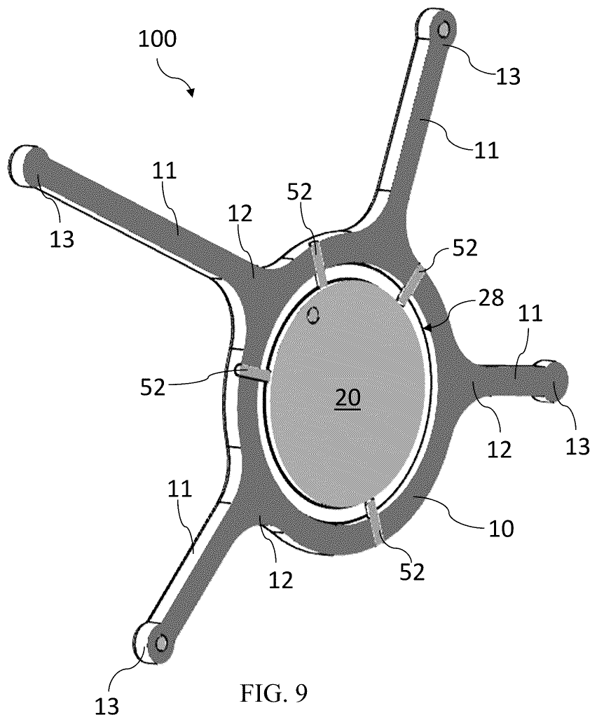

FIG. 9 illustrates a cross-sectional view of the tracker 100 taken through the line 9-9 of FIG. 6. As illustrated, the dowels 52 can be seen interfacing and engaging with the circumferential groove 28 of the center 20. The dowels 52 may secure the center 20 within the frame 10 and the central recess 15. The circumferential groove 28 may be continuous about a circumference of the center 20, such that the dowels 52 slide within the circumferential groove 28 during movement of the center 20 (such as from a first discrete position to a second discrete position). While these figures illustrate one method (rotation) of allowing the center to move relative to the frame, other methods of moving the center relative to the frame can also be used to allow the instrument-type marker to have different positions for identifying instrument types.

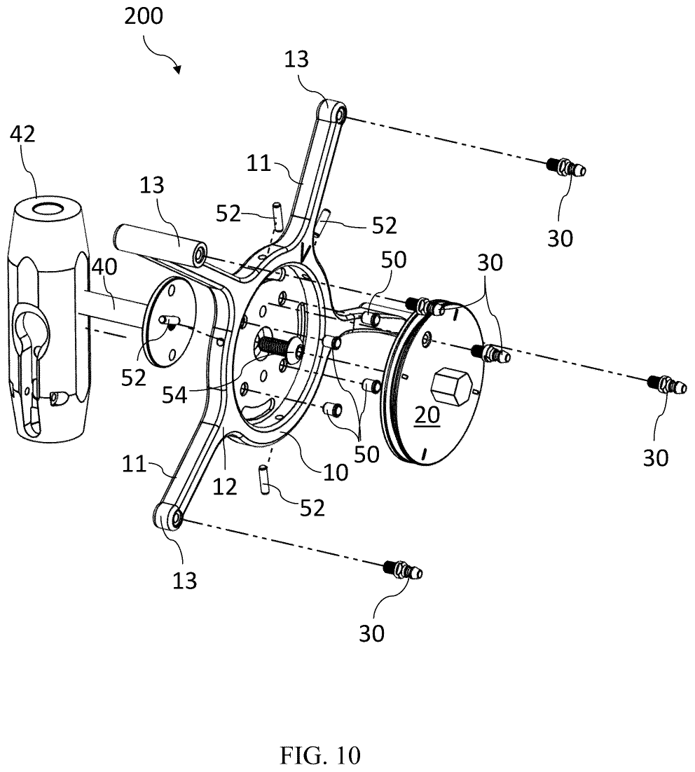

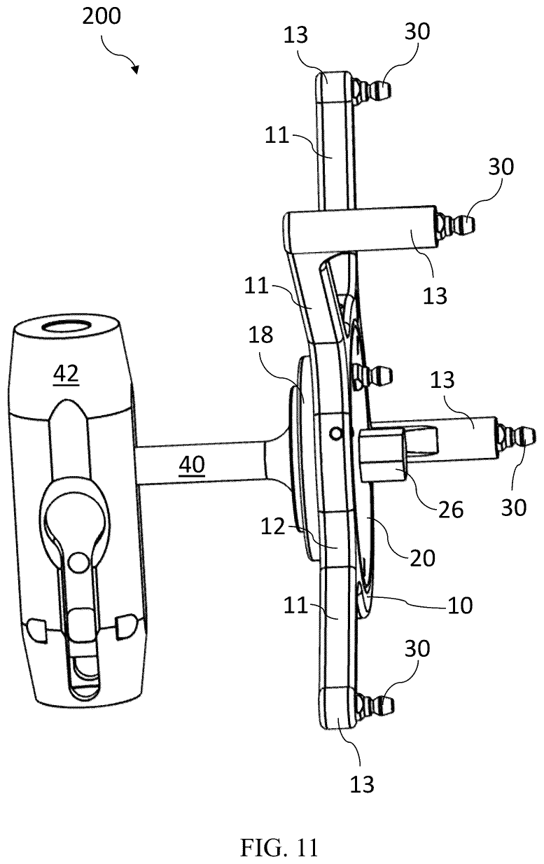

FIG. 10 illustrates an exploded view and FIG. 11 illustrates a side view of a tracking system 200, including the tracker 100 of FIGS. 1 through 9, and a mechanism for connecting the tracker to a surgical instrument. Specifically, the tracker 100 is connectable to a body 42, where the body 42 is further connectable to a surgical instrument. The tracker 100 is connectable to the body 42 through a post 40. As seen in FIG. 11, the post 40 is connectable to the platform 18 at the back 19b of the frame 10 of the tracker 100. The post 40 may be secured to the platform 18 and the frame 10 through one or more fasteners. For example, the post 40 may be secured to the frame 10 through a screw 54, which may be a button head screw. Any appropriate type of fastener may be used to secure the post 40 to the frame 10.

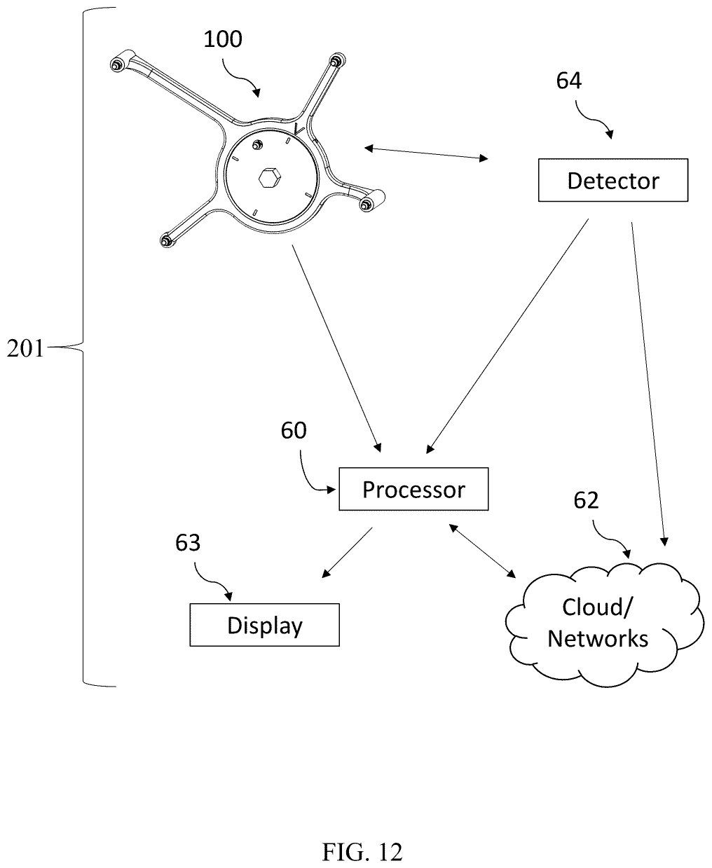

FIG. 12 schematically illustrates another embodiment of a tracking system 201. The tracking system 201 may include the tracker 100 (which may be connected to the body 42 through the post 40), a detector or sensor 64, a processor 60, a display 63, and one or more networks 62. The detector or sensor 64 is for detecting the tracking markers 30 (e.g., the surgical instrument positioning trackers 30 and the instrument-type marker 30) and a position of the tracking markers 30. The detector or sensor 64 may be a camera, a thermal imaging device, a radiofrequency (RF) detector, or any appropriate detector or sensor 64 for detecting the tracking markers 30. The detector or sensor 64 may detect a presence and position of the tracking markers 30.

The detector or sensor 64 is in communication with the processor 60 and communicates the presence and position of the tracking markers 30. Specifically, the processor 60 receives signals from the detector or sensor 64 that correspond to the position of the tracking markers 30. Based on the signals received, the processor 60 determines (i) a position of the surgical instrument attached to the tracker 100 and (ii) a type of the surgical instrument attached to the tracker 100. The detector or sensor 64 and the processor 60 may be in communication with a display 63 for displaying the position and type of the surgical instrument. The processor 60 may also aid in navigation of the surgical instrument based on the determined position of the surgical instrument.

The processor 60 may include any appropriate and necessary modules for receiving signals from the detector or sensor 64, for processing the received signals, and for determining position and type of the surgical instrument based on the received and processed signals. For example, the processor 60 may include communications modules (e.g., Bluetooth, Wi-Fi, etc.), memory and storage modules, one or more microprocessor units, as well as any other appropriate module.

The processor 60 may also include software, firmware, and/or programming that can be set to any pre-determined desired angle for spacing the discrete positions of the center 20 and the instrument-type tracking marker 30 apart. The software or programming can also be set to any pre-determined desired threshold or tolerance value between the discrete positions, such that the center 20 and/or the instrument-type tracking marker 30 may be within the threshold value of the discrete position and be detected at the discrete position. For example, the pre-determined desired threshold value may be 10-degrees. When the center 20 and/or the instrument-type tracking marker 30 are within 10-degrees of a first discrete position, the center 20 and/or the instrument-type tracking marker 30 will be detected at the first discrete position. Other threshold values, positions, instrument types, etc., can be programmed into software for use with the system.

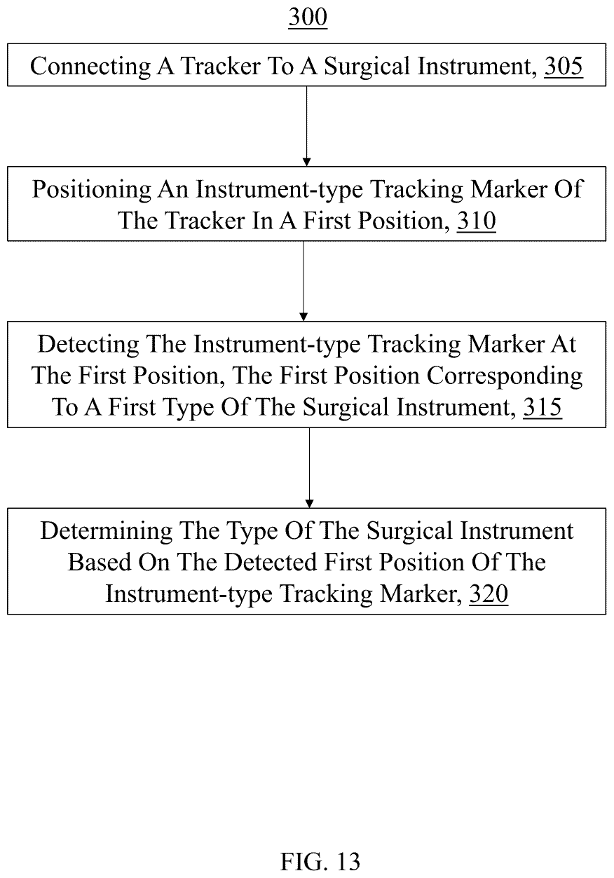

FIG. 13 is a flowchart of one example method 300 of identifying a type of surgical instrument in navigated or robotic-assisted surgery. The method 300 may include connecting a tracker to a surgical instrument, at 305, and positioning an instrument-type tracking marker of the tracker in a first position, at 310. The tracker may be the tracker 100 of FIGS. 1 through 12. The method 300 may also include detecting the instrument-type tracking marker at the first position, the first position corresponding to a first type of the surgical instrument, at 315. Further, the method 300 may include determining the type of the surgical instrument based on the detected first position of the instrument-type tracking marker, at 320.

Connecting a tracker to a surgical instrument may include connecting the surgical instrument to a body connected to the tracker, such as body 42 connected to a frame 10 of the tracker 100. Positioning an instrument-type tracking marker of the tracker in a first position may include rotating a center of the tracker to the first position, where the instrument-type tracking member is disposed on the center. The center may be secured in the first position through a detent and pin, magnetically, or another appropriate securement mechanism. Detecting the instrument-type tracking marker at the first position may include visually capturing, by a camera, the tracking marker at the first position.

The method 300 may further include connecting a second surgical instrument to the tracker and positioning the instrument-type tracking marker of the tracker in a second position different than the first position. The method 300 may additionally include detecting the instrument-type tracking marker at the second position and determining a second type of the surgical instrument based on the detected second position of the instrument-type tracking marker. Further, the method 300 may include communicating the first type of the surgical instrument to a processor and tracking a position of the surgical instrument.

Tracking a position of the surgical instrument may include detecting a position of a first instrument-positioning tracker disposed at an end of a first arm of the tracker. Tracking the position may also include detecting a position of a second instrument-positioning tracker disposed at an end of a second arm of the tracker and determining the position of the surgical instrument based on the detected position of the first instrument-positioning tracker and the second instrument-positioning tracker.

### Embodiments

Embodiment 1: A tracker for use in navigated or robotic-assisted surgery, the tracker comprising:
a frame connectable to a surgical instrument;
at least one arm extending from the frame from a first end to a second end, the at least one arm having a surgical instrument positioning tracking marker at the second end, the surgical instrument positioning tracking marker for determining a position of the surgical instrument; and
an instrument-type tracking marker disposed on the frame, the instrument-type tracking marker for determining a type of the surgical instrument.

Embodiment 2: The tracker of Embodiment 1, wherein the frame comprises a center having a plurality of set positions about the frame, each set position of the plurality of set positions corresponding to a type of the surgical instrument connectable to the frame.

Embodiment 3: The tracker of Embodiment 2, wherein the plurality of set positions about the frame comprises four (4) discrete set positions.

Embodiment 4: The tracker of Embodiment 2 or Embodiment 3 wherein the plurality of set positions are spaced a distance from each other of about 90 degrees.

Embodiment 5: The tracker of any one of Embodiments 2-4, wherein the center is rotatable between each of the plurality of set positions.

Embodiment 6: The tracker of any one of Embodiments 1 through 5, wherein a position of the instrument-type tracking marker about the frame corresponds to the type of the surgical instrument.

Embodiment 7: The tracker of any one of Embodiments 1 through 6, wherein the at least one arm comprises a plurality of arms extending from the frame from a first end to a second end, with each arm of the plurality of arms having a surgical instrument positioning tracking marker at the second end.

Embodiment 8: The tracker of any one of Embodiments 1 through 7, further comprising a center in association with the frame, the center comprising an engagement mechanism for moving the instrument-type tracking marker from a first position to a second position different than the first position.

Embodiment 9: The tracker of Embodiment 8, wherein the engagement mechanism comprises a handle to be grasped by a user.

Embodiment 10: The tracker of Embodiment 8 or Embodiment 9, wherein the engagement mechanism comprises a nut to be engaged by a driver.

Embodiment 11: A system for use in a navigated or robotic-assisted surgical procedure, the system comprising:
a tracker connectable to a surgical instrument for use in the surgical procedure, the tracker for identifying a type of the surgical instrument and for tracking a position of the surgical instrument during the surgical procedure, the tracker comprising:
a frame connectable to the surgical instrument, at least one arm extending from the frame from a first end to a second end, the at least one arm having a first tracking marker at the second end, the first tracking marker for determining a position of the surgical instrument, and a second tracking marker in connection with the frame, the second tracking marker having a plurality of positions relative to the frame, the second tracking marker for determining a type of the surgical instrument;

a processor in communication with a sensor configured to track the tracker, the processor for receiving signals from the sensor, and the processor for determining the type of the surgical instrument and the position of the surgical instrument during the surgical procedure; and
a display in communication with the processor, the display for displaying the type of the surgical instrument and the position of the surgical instrument during the surgical procedure.

Embodiment 12: The system of Embodiment 11, wherein the processor determines the position of the surgical instrument through signals received from the sensor based on the sensor's tracking of the first tracking marker of the at least one arm.

Embodiment 13: The system of either one of Embodiment 11 or 12, wherein the processor determines the type of the surgical instrument through signals received from the sensor based on the sensor's tracking of the second tracking marker of the frame.

Embodiment 14: The system of any one of Embodiments 11 through 13,wherein the sensor comprises a camera for tracking the first tracking marker of the at least one arm and the second tracking marker of the frame.

Embodiment 15: The system of Embodiment 14, wherein the camera is in communication with the processor and the display.

Embodiment 16: A method for identifying a type of surgical instrument in navigated or robotic-assisted surgery, the method comprising:
connecting a tracker to a surgical instrument;
positioning an instrument-type tracking marker of the tracker in a first position;
detecting the instrument-type tracking marker at the first position, the first position corresponding to a first type of the surgical instrument; and
determining the type of the surgical instrument based on the detected first position of the instrument-type tracking marker.

Embodiment 17: The method of Embodiment 16, wherein positioning an instrument-type tracking marker of the tracker in the first position comprises rotating a center of the tracker to the first position, the instrument-type tracking member disposed on the center; and securing the center in the first position.

Embodiment 18: The method of Embodiment 17, wherein securing the center in the first position comprises engaging a detent with a pin.

Embodiment 19: The method of Embodiment 17, wherein securing the center in the first position comprises magnetically securing the center in the first position.

Embodiment 20: The method of any one of Embodiments 16 through 19, wherein detecting the instrument-type tracking marker at the first position comprises visually capturing, by a camera, the tracking marker at the first position.

Embodiment 21: The method of any one of c Embodiments 16 through 20, further comprising:
connecting a second surgical instrument to the tracker;
positioning the instrument-type tracking marker of the tracker in a second position different than the first position;
detecting the instrument-type tracking marker at the second position; and
determining a second type of the surgical instrument based on the detected second position of the instrument-type tracking marker.

Embodiment 22: The method of any one of Embodiments 16 through 21, further comprising communicating the first type of the surgical instrument to a processor and tracking a position of the surgical instrument.

Embodiment 23: The method of Embodiment 22, wherein tracking a position of the surgical instrument comprises:
detecting a position of a first instrument-positioning tracker disposed at an end of a first arm of the tracker;
detecting a position of a second instrument-positioning tracker disposed at an end of a second arm of the tracker; and
determining the position of the surgical instrument based on the detected position of the first instrument-positioning tracker and the second instrument-positioning tracker.

### Additional Terms and Definitions

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It should also be noted that some of the embodiments disclosed herein may have been disclosed in relation to a particular tracking marker (e.g., an optical tracking marker); however, other markers (e.g., lights, thermal markers, RF markers, magnetic, etc.) are also contemplated. Tracking markers can be any suitable shape, size, etc., desired.

In one embodiment, the terms “about” and “approximately” refer to numerical parameters within 10% of the indicated range. The terms “a,” “an,” “the,” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the author(s) of this disclosure for carrying out the embodiments disclosed herein. Of course, variations on these described embodiments will become apparent to those of ordinary skill in the art upon reading the foregoing description. The author(s) expects skilled artisans to employ such variations as appropriate, and the author(s) intends for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of this disclosure so claimed are inherently or expressly described and enabled herein.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A tracker for use in navigated or robotic-assisted surgery, the tracker comprising:
a frame connectable to a surgical instrument;
an arm extending from the frame from a first end to a second end, the arm having a surgical instrument positioning tracking marker at the second end, the surgical instrument positioning tracking marker for determining a position of the surgical instrument; and
an instrument-type tracking marker disposed on the frame, the instrument-type tracking marker for determining a type of the surgical instrument.

2. The tracker of claim 1, wherein the frame comprises a center having a plurality of set positions about the frame, each set position of the plurality of set positions corresponding to a type of the surgical instrument connectable to the frame.

3. The tracker of claim 2, wherein the plurality of set positions about the frame comprises four (4) discrete set positions.

4. The tracker of claim 3 wherein the plurality of set positions are spaced a distance from each other of about 90 degrees.

5. The tracker of claim 2, wherein the center is rotatable between each of the plurality of set positions.

6. The tracker of claim 1, wherein a position of the instrument-type tracking marker about the frame corresponds to the type of the surgical instrument.

7. The tracker of claim 1, wherein the tracker comprises a plurality of arms extending from the frame from a first end to a second end, with each arm of the plurality of arms having a surgical instrument positioning tracking marker at the second end.

8. The tracker of any one of claim 1, further comprising a center in association with the frame, the center comprising an engagement mechanism for moving the instrument-type tracking marker from a first position to a second position different than the first position.

9. The tracker of claim 8, wherein the engagement mechanism comprises one or more of: a handle to be grasped by a user, and a nut to be engaged by a driver.

10. A system for use in a navigated or robotic-assisted surgical procedure, the system comprising:
a tracker connectable to a surgical instrument for use in the surgical procedure, the tracker for identifying a type of the surgical instrument and for tracking a position of the surgical instrument during the surgical procedure, the tracker comprising:
a frame connectable to the surgical instrument,
at least one arm extending from the frame from a first end to a second end, the at least one arm having a first tracking marker at the second end, the first tracking marker for determining a position of the surgical instrument, and
a second tracking marker in connection with the frame, the second tracking marker having a plurality of positions relative to the frame, the second tracking marker for determining a type of the surgical instrument;
a processor in communication with a sensor configured to track the tracker, the processor for receiving signals from the sensor, and the processor for determining the type of the surgical instrument and the position of the surgical instrument during the surgical procedure; and
a display in communication with the processor, the display for displaying the type of the surgical instrument and the position of the surgical instrument during the surgical procedure.

11. The system of claim 10, wherein the processor determines the position of the surgical instrument through signals received from the sensor based on the sensor's tracking of the first tracking marker of the at least one arm.

12. The system of either one of claim 10, wherein the processor determines the type of the surgical instrument through signals received from the sensor based on the sensor's tracking of the second tracking marker of the frame.

13. The system of claim 10, wherein the sensor comprises a camera for tracking the first tracking marker of the at least one arm and the second tracking marker of the frame.

14. The system of claim 13, wherein the camera is in communication with the processor and the display.

15. A method for identifying a type of surgical instrument in navigated or robotic-assisted surgery, the method comprising:
connecting a tracker to a surgical instrument;
positioning an instrument-type tracking marker of the tracker in a first position;
detecting the instrument-type tracking marker at the first position, the first position corresponding to a first type of the surgical instrument; and
determining the type of the surgical instrument based on the detected first position of the instrument-type tracking marker.

16. The method of claim 15, wherein positioning an instrument-type tracking marker of the tracker in the first position comprises rotating a center of the tracker to the first position, the instrument-type tracking member disposed on the center; and securing the center in the first position.

17. The method of claim 16, wherein securing the center in the first position comprises one or more of: engaging a detent with a pin, and/or magnetically securing the center in the first position.

18. The method of claim 15, wherein detecting the instrument-type tracking marker at the first position comprises visually capturing, by a camera, the tracking marker at the first position.

19. The method of claim 15, further comprising:
connecting a second surgical instrument to the tracker;
positioning the instrument-type tracking marker of the tracker in a second position different than the first position;
detecting the instrument-type tracking marker at the second position; and
determining a second type of the surgical instrument based on the detected second position of the instrument-type tracking marker.

20. The method of claim 15, further comprising communicating the first type of the surgical instrument to a processor and tracking a position of the surgical instrument.

21. The method of claim 20, wherein tracking a position of the surgical instrument comprises:
detecting a position of a first instrument-positioning tracker disposed at an end of a first arm of the tracker;
detecting a position of a second instrument-positioning tracker disposed at an end of a second arm of the tracker; and
determining the position of the surgical instrument based on the detected position of the first instrument-positioning tracker and the second instrument-positioning tracker.
